(19) United States
(12) Patent Application Publication
(10) Pub. No.: US 2008/0304729 A1
(43) Pub. Date: Dec. 11, 2008

(54) Method and Apparatus for Continuous Imaging by Ultrasound Transducer System

(75) Inventor: Michael Peszynski, Newburyport, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/912,588

(22) PCT Filed: Apr. 20, 2006
(86) PCT No.: PCT/IB2006/051226

§ 371 Date: Jul. 7, 2008

Related U.S. Application Data
(60) Provisional application No. 60/674,493, filed on Apr. 25, 2005.

(51) Int. Cl. A61B 8/00

(52) U.S. Cl. 382/131

(57) ABSTRACT

A low profile large aperture matrix based ultrasound transducer fixably attached to the human body by a disposable pad and is used to image the human anatomy. The image tuning and field of view is controlled remotely by inputs to the ultrasound imaging system.

### BACKGROUND OF THE INVENTION

1. Field of the Invention

The present invention relates to a method and apparatus for providing a continuous imaging by an ultrasound transducer system. In particular the present invention relates to a method and apparatus for ultrasound imaging that controls the tuning and positioning of scan lines generated by an array without the need for a manual transducer manipulation.

2. The Prior Art

For transthoracic imaging ultrasound transducers are typically hand held against the chest or abdomen.

In order to provide a continuous imaging of human anatomy for evaluation or therapy, an ultrasound transducer needs to be positioned and held in with very good acoustic coupling and precisely aligned with the targets of interest. Remote transducers have been described by Chanderatna (5598845) and Clancy (5022410) but in both cases mechanical adjustment of the transducer assembly relative to the human anatomy is required for image acquisition. It would be desirable to develop a methodology and an apparatus that permits remote transducer usage without the need for manual adjustment.

### SUMMARY OF THE INVENTION

The invention described here is a low profile large aperture matrix based ultrasound transducer fixably attached to the human body by a disposable pad and is used to image the human anatomy. The image tuning and field of view is controlled remotely by inputs to the ultrasound imaging system.

The matrix array pad applied transducer described here removes the need for mechanical adjustment by utilizing electronic control of scan lines that are positioned by the user controlling the ultrasound imaging system so that it is no longer necessary to manipulate the imaging transducer.

### BRIEF DESCRIPTION OF THE DRAWINGS

FIG. 1 is a block diagram of the present invention showing a matrix array sensor assembly controlled by a phased array ultra sound imaging system and a disposable pad is attached to the transducer housing and acoustically coupled to the array;

FIG. 2 illustrates the patch of FIG. 1 being attached to a patient's body in an area of interest;

FIG. 3 is an alternative embodiment to FIG. 2 showing multiple patches attached to multiple areas of interest;

FIGS. 4A and 4B show an alternative patch—a reusable matrix array patch in which the patch is a reusable patch shown in top and side views, respectively;

FIGS. 5A and 5B are top and side views, respectively of the disposable patch of FIG. 1;

FIGS. 6A and 6B illustrate a matrix array patch applied to a patient's body for imaging where imaging is cannot be visualized due to a rib's shadowing;

FIGS. 7A and 7B illustrate how the present invention over comes the problems of imaging in FIGS. 6A and 6B due to rib shadowing; and

FIG. 8 illustrates the phased array ultra sound imaging system control panel of the present invention and the controls for adjusting the imaging by the transducer patch including removing rib shadowing as described in FIGS. 6A, 6B, 7A and 7B.

### DETAILED DESCRIPTION OF THE PREFERRED EMBODIMENTS

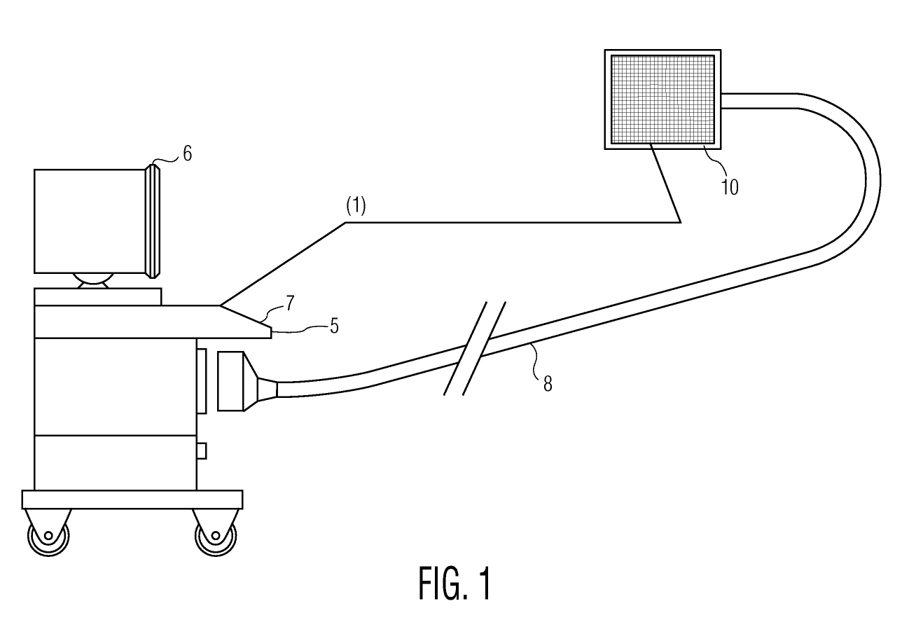

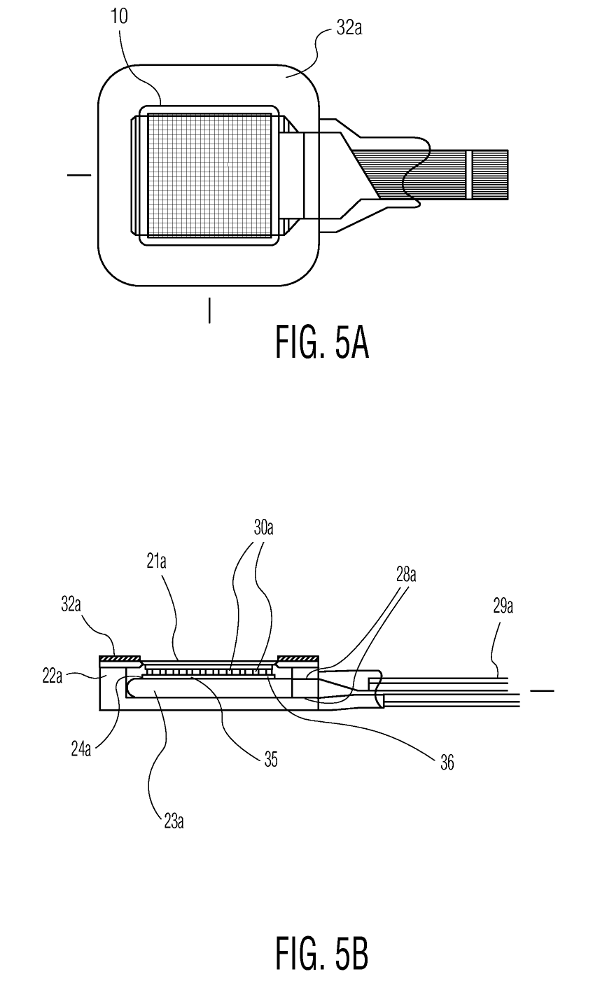

Referring now to the drawings of FIGS. 1-8, a low profile large aperture matrix array sensor assembly controlled by a phased array ultrasound imaging system is shown in FIG. 1. The array is held captive in a low profile rigid housing and connected to the imaging system by conventional transducer wiring (although a wireless connection could be any commercially known wireless technology such as but not limited to Bluetooth® technology). A matrix patch lo can be formed as a disposable pad and made of suitable low acoustic loss material such as silicon or equivalent is attached to the transducer housing and acoustically coupled to the array with ultrasound gel. The disposable pad, described in more detail in FIGS. 5A and 5B, is then attached to the human body in the area of interest with adhesive on its perimeter and acoustically couple to the body with ultrasonic gel.

Images obtainable from the matrix array include both standard 2D phased or linear array formats as well as 3D real-time volume imaging as described in U.S. Pat. No. 6,679,849. The images may be tuned and manipulated electronically from the ultrasound imaging system. Keyhole imaging may be used for example to image in between ribs if the array pad was inadvertently placed over one during cardiac imaging. Multiple transducers may be envisioned running on the same system depending upon the clinical imaging requirements at hand.

The low profile matrix array may be of a Capacitive Micromachined Ultrasound Transducer (CMUT)—see U.S. Pat. No. 6,585,653, a Piezoelectric Micromachined Ultrasound Transducer (PMUT)—see U.S. Pat. No. 6,659,954, micro machined ultrasound transducer construction, or of a piezo based construction as described in U.S. Pat. No. 6,679,849. The CMUT would be manufactured using standard integrated circuit processes where capacitively coupled micro machined drums would create the acoustic beams. The ASIC is integrally fabricated as part of the CMUT. The PMUT would be manufactured using integrated circuit processes where piezoelectric elements would create the acoustic beams. The ASIC is fabricated first then the piezo material would be doped afterwards.

The matrix array assembly would be attached to a rigid transducer housing and preferably a low profile rigid housing, using standard techniques. The acoustic interface materials are known in the art. A low loss pad whose thickness is sufficient to absorb minor changes in human body contours would be manufactured as a disposable such that it could be attached to and later removed from the transducer housing and applied with acoustic gel to insure very good acoustic coupling between transducer and pad. A release film would be applied at the perimeter of the human to pad adhesive interface. Once the transducer position of interest was determined acoustic gel would be applied to the pad and the release film removed and the transducer applied to the patient imaging area. Once good acoustic contact was obtained all imaging control would be input at the imaging system without the need to manipulate the transducer array.

The imaging system 5 can be phased array ultrasound imaging system 5 for controlling the array 10 so that images from the array 10 include both standard 2D phased and linear array formats as well as 3D real-time imaging as described in U.S. Pat. No. 6,679,849. The ultra sound imaging system 5 could be any suitable commercially known ultrasound imaging system such as but not limited to Philip's Sonos 7500. The images may be tuned and manipulated electronically from the ultrasound imaging system 5. This system includes a monitor 6 and a console control 7. The ultra sound imaging system 5 is connected by wire 8 as shown in FIG. 1 or wirelessly to the ultra sound transducer 10.

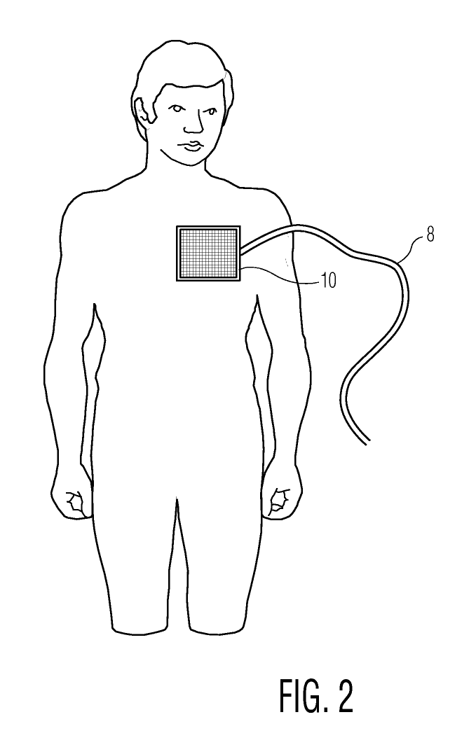

The matrix ultrasound transducer can be formed as a patch that adheres to a portion of patient's for imaging such as cardiac imaging as shown in FIG. 2. The wire 8 transmits the images to the ultra sound imaging system 5 for viewing on the monitor 6.

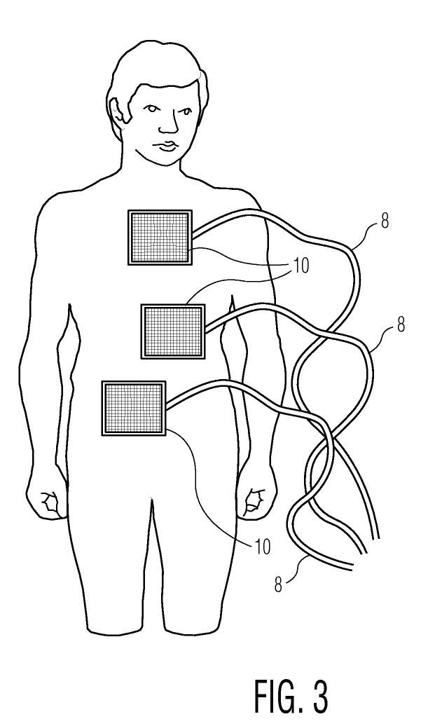

FIG. 3 is an alternative embodiment in which several matrix ultra sound transducer patches are affixed to a patient. Such multiple array patches might prove useful for cardiac monitoring by locating the patches over standard cardiac imaging windows on the patient's body such as the suprasternal, parasternal, and subcostal areas. It is understood that this embodiment is not limited to cardiac imaging but may be used whenever placement of multiple patches may prove useful perhaps when monitoring a pregnant woman and her fetus.

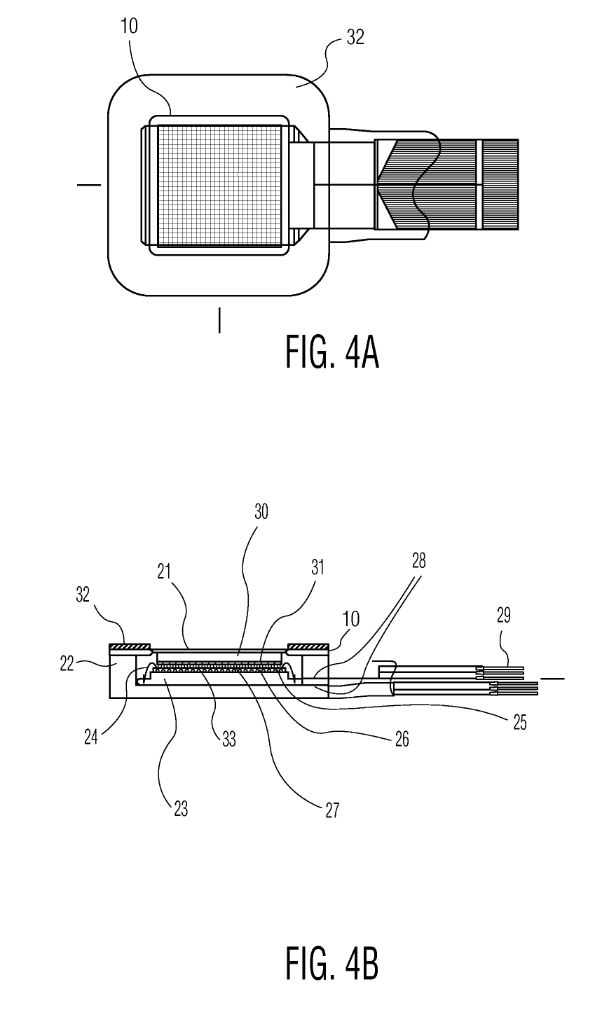

FIGS. 4 A and 4 B illustrate a reusable patch for the matrix array 10 which matrix array is described in U.S. Pat. No. 6,685,647 using a de-matching layer for low profile assembly. The reusable matrix array is formed of a standard piezoelectric based acoustic stack connected through a ball grid or equivalent interconnect to an ASIC.

FIG. 4A shows the top view of the reusable patch 10. FIG. 4B shows the sectional view illustrating the construction of the matrix array reusable patch 10. As seen in FIG. 4B there is an acoustic window 21; acoustic matching layers 30; a piezoelectric element 31; a removable double-sided grade tape 32; a plastic housing 22; a microbeamforming silican ASIC 25; an acoustic de-matching layer 26; a stud bump or ball grid array in conductive epoxy used to connect array acoustic elements to microbeamforming ASIC 27 and therefore provides conductivity between the two; an epoxy backfill 33 that isolates the individual conductive elements from each other; a heat sink bonded to ASIC and flexible circuit 23; a wire band ASIC to flexible circuit interconnect 24; flexible circuits 28; and a coax cable array 29.

FIGS. 5 A and 5 B illustrate a disposable patch for the matrix array 10 which matrix array is described in U.S. Pat. No. 6,685,647 using a de-matching layer for low profile assembly. FIG. 5A shows the top view of the disposable patch 10. FIG. 5B shows the sectional view illustrating the construction of the matrix array disposable patch 10. As seen in FIG. 5B there is an acoustic window 21a; a microbeamforming ASIC with active CMUT or PMUT acoustic matrix array integrally attached 30a; a permanent double sided medical grade tape affixed in a plastic housing 32a, a plastic housing 22a; a heat sink bonded to ASIC and flexible circuit 23a; a wire band ASIC to flexible circuit interconnect 24a; flexible circuits 28a; an acoustic de-matching layer 35; microbeamforming silicon ASIC 36; and micro flat ribbon cable assembly 29a. The patch can be made of silicon or equivalent material with adhesive around its perimeter and acoustically coupled to a patient's body in the area of interest with ultrasonic gel.

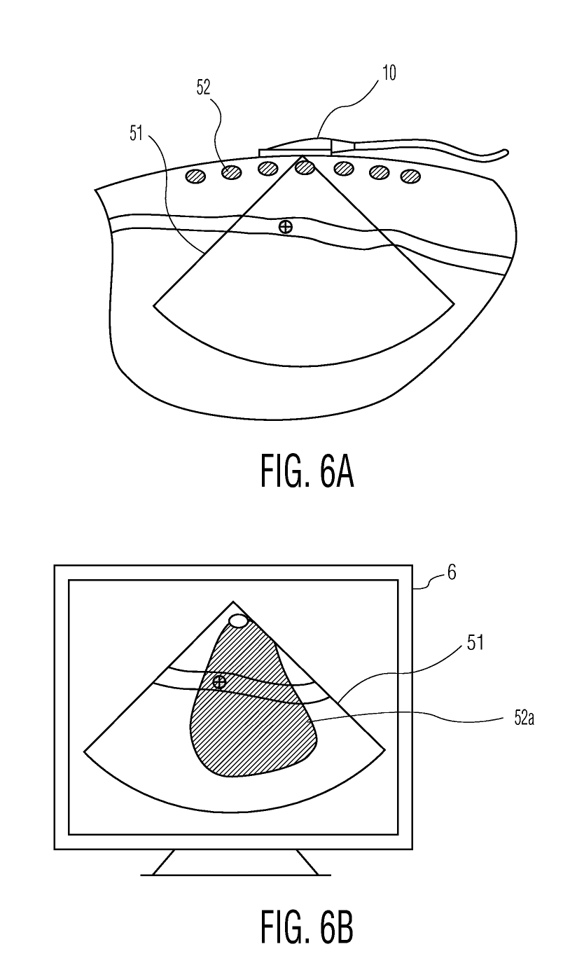

FIGS. 6A and 6B illustrate the problem with ultra sound imaging and 3D ultrasound imaging in an imaging mode with a matrix patch that is positioned over an imaging target. The present invention provides for imaging and this includes 2D or 3D imaging. The present invention provides for a novel solution such problems by first providing a system and method for imaging over one or more imaging targets having an obstruction without the need for any mechanical adjustment of the matrix patch but by remote operation of the controls on the ultrasound imaging system 5. In the example presented rib shadowing is caused by one or more ribs but it is understood that the invention is not limited to this one obstruction or reason for imaging as described herein. Second, the present invention provides for positioning the matrix patch 10 over one or more targets to visualize at least one or more targets by repositioning the sector scans using the controls on the ultrasound imaging system 5. This makes it possible to visualize multiple targets remotely with the ultrasound imaging system 5.

Under these conditions the imaging target underneath the ribs cannot be visualized because of the rib shadowing acoustic scan lines 52a. As seen in FIG. 6A the matrix array patch 10 is adhered to a patient's body with acoustic gel applied between the transducer and the patient. A 2D scan 51 is produced using a partial aperture available in the matrix array patch 10. However a patient's ribs 52 blocks access to acoustic scan lines.

FIGS. 6A and 6B illustrate the problem with ultra sound imaging and also with 3D ultrasound imaging in a 2D imaging mode with a matrix patch that is positioned over an imaging target underneath the ribs. This illustration is only one example of an application of the present invention and is not intended to be limited thereto. The present invention, as noted previously, is utilized for sector scanning, volume scanning, and elimination of obstructions while imaging and imaging remotely in more than one area of interest of a patient's body. Turning now to the specific example where rib shadowing provides an obstruction, under these conditions the imaging target underneath the ribs cannot be visualized because of the rib shadowing acoustic scan lines 52a. As seen in FIG. 6A the matrix array patch 10 is adhered to a patient's body with acoustic gel applied between the transducer and the patient. A 2D scan 51 is produced using a partial aperture available in the matrix array patch 10. However a patient's ribs 52 blocks access to acoustic scan lines.

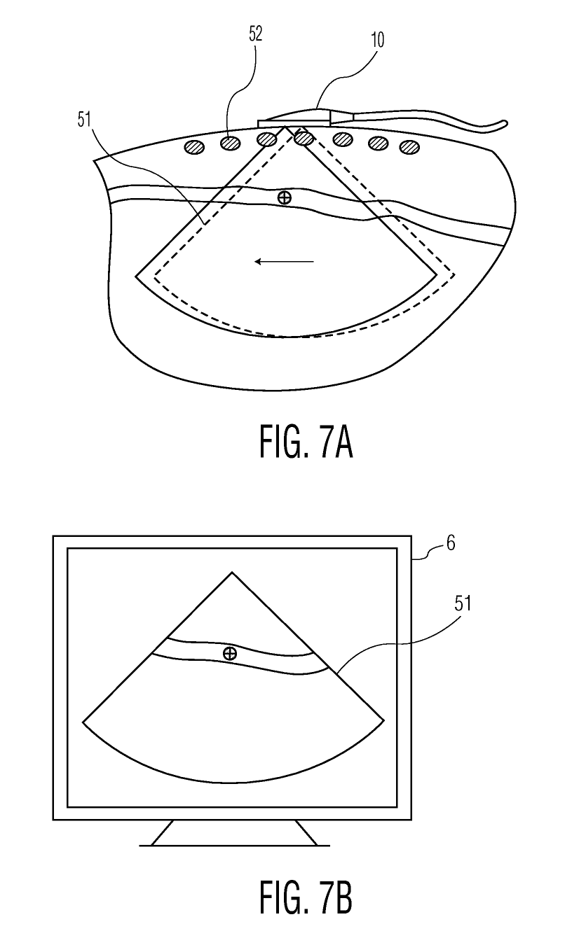

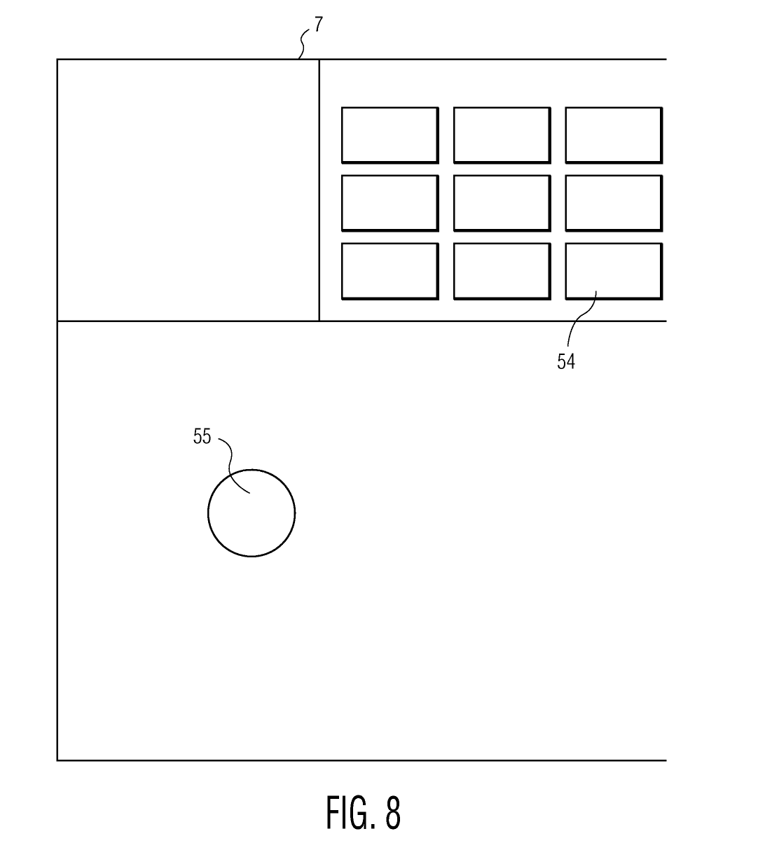

The present invention provides a solution to this problem as shown in FIGS. 7A, 7B and FIG. 8.

In FIGS. 7A and 7B the matrix array patch 10 is applied with the acoustic gel to the patient's body with the acoustic gel being applied between the transducer and the patient. Again the patient's ribs 52 block access to acoustic scan lines. The 2D sector scan 51a is repositioned from the imaging system's 5 console 7 by utilizing the console controls touch screen keys 54 and the trackball 55.

The trackball 55 is rotated accordingly to scroll the image to the left or to the right in order to position the image with the rib out of the way. The soft key controls 54 also provide various movement of the image as indicated in FIG. 8 such as tilt, elevation, biplane rotate, etc. for movement of the image from the rib seen in FIG. 7B. The 3D ultrasound system operates in a 2D imaging mode with a matrix patch 10 that is positioned over an imaging target and can visualize the image by repositioning sector scanning horizontally using a remote system control 5.

As stated previously the controls on these consoles can be used to image targets having any obstructions or for visualizing more than one target and the present invention is not limited to any one particular use.

The present invention provides for ultrasound imaging without the need for repositioning the matrix array patch and also for removing obstructions such as rib shadowing remotely.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. A continuous imaging ultrasound transducer and system, comprising:
a low profile transducer, said transducer including a large aperture matrix array;
an ultrasound imaging system that controls image tuning and positioning of scan lines generated by said matrix array; and
said matrix array including a pad made of a low acoustic loss material and being sufficiently larger than an actual imaging aperture so that patient placement is not critical and imaging position may be manipulated remotely by said imaging system without any mechanical adjustment of said transducer.

2. The transducer and system according to claim 1 further comprising:
said ultrasound imaging system in an imaging mode with said matrix patch being positioned over at least one imaging target having an obstruction to visualize an image by repositioning sector scanning using controls on said ultrasound imaging system to remove said obstruction from said image.

3. The transducer and system according to claim 2 wherein said imaging mode is a 2D imaging mode.

4. The transducer and system according to claim 2 wherein said imaging mode is a 3D imaging mode.

5. The transducer and system according to claim 1 wherein said matrix patch is positioned over at least one imaging target having an obstruction to visualize an image by repositioning sector scanning using said controls horizontally.

6. The transducer and system according to claim 1 wherein said matrix patch is positioned over at least one imaging target having an obstruction to visualize an image by repositioning sector scanning using said controls vertically.

7. The transducer and system according to claim 1 wherein said matrix patch is positioned over at least one imaging target having an obstruction to visualize an image by repositioning sector scanning using said controls to rotate.

8. The transducer and system according to claim 1 wherein said matrix patch is positioned over at least one imaging target having an obstruction to visualize an image by repositioning sector scanning using said controls to tilt.

9. The transducer and system according to claim 1 wherein said matrix patch is positioned over at least one imaging target having an obstruction to visualize an image by repositioning sector scanning using said controls to move the image along its x axis.

10. The transducer and system according to claim 1 wherein said matrix patch is positioned over at least one imaging target having an obstruction to visualize an image by repositioning sector scanning using said controls to move the image along its y axis.

11. The transducer and system according to claim 2 wherein said ultrasound imaging system in said imaging mode with said matrix patch is positioned over at least one imaging target to visualize an image by repositioning sector scanning using controls on said ultrasound imaging system to remove rib shadowing from said image.

12. The transducer and system according to claim 11 wherein said controls on said ultra imaging system includes a trackball for scrolling said image to a left or a right of said rib in order to position the image with the rib out of the way and soft key controls 54 on said ultra imaging system to provide various movement of the image such as tilt, elevation, biplane rotate, etc. for movement of said image from said rib.

13. The transducer and system according to claim 1 further comprising: said ultrasound imaging system in an imaging mode with said matrix patch being positioned over at least one target to visualize at least one target by repositioning sector scanning using controls on said ultrasound imaging system.

14. The transducer and system according to claim 1 wherein said pad is a disposable pad.

15. The transducer and system according to claim 1 wherein said pad is a reusable pad.

16. The transducer and system according to claim 1 wherein said matrix array and said ultrasound imaging system are connected transducer wiring pad.

17. The transducer and system according to claim 1 wherein said matrix array and said ultrasound imaging system are connected by wireless technology.

18. The transducer and system according to claim 5 wherein said wireless technology is Bluetooth® technology.

19. The transducer and system according to claim 1 wherein said matrix array is formed as multiple pads for imaging.

20. The transducer and system according to claim 1 wherein said matrix array is a low profile large aperture profile matrix array sensor assembly.

21. The transducer and system according to claim 21 wherein said array is made of CMUT.

22. The transducer and system according to claim 21 wherein said array is made of PMUT.

23. The transducer and system according to claim 21 wherein said array is made of a micro machined ultrasound transducer construction.

24. The transducer and system according to claim 21 wherein said array is made of a piezo based construction.

25. The transducer and system according to claim 21 wherein said array is held in a low profile rigid housing and connected to said imaging system by transducer wiring.

26. The transducer and system according to claim 21 wherein said array is held in a low profile rigid housing thereby providing a housing for said transducer and connected to said imaging system by wireless technology.

27. The transducer and system according to claim 27 wherein said wireless technology is Bluetooth® technology.

28. The transducer and system according to claim 21 wherein said array is attached to a rigid housing for said transducer and acoustically coupled to said array with an ultrasound gel.

29. The transducer and system according to claim 29 wherein said pad is attached to a patient's body in an area of interest with adhesive on a perimeter of said pad and acoustically coupled said patient's body with said ultrasound gel.

30. The transducer and system according to claim 21 wherein said imaging system is a phased array ultrasound imaging system and said phased array imaging system controls said array wherein images obtained from said array include both standard 2D phased array formats and 2D linear array formats and also 3D real-time volume images.

31. A method for providing continuous imaging ultrasound, the steps comprising:
generating scan lines by means of a large matrix array of a low profile transducer;
controlling image turning and positioning of scan lines generated by a matrix array by means of an ultrasound imaging system; and
providing said matrix array includes a pad made of a low acoustic loss material and being sufficiently larger than an actual imaging aperture so that patient placement is not critical and imaging position may be manipulated remotely by said imaging system without any mechanical adjustment of said transducer.

32. The method according to claim 32 further comprising the steps of:
positioning said ultrasound imaging system in an imaging mode with said matrix patch over at least one imaging target having an obstruction to visualize an image by repositioning sector scanning using controls on said ultrasound imaging system to remove said obstruction from said image.

33. The method according to claim 32 wherein said imaging mode is a 2D imaging mode.

34. The method according to claim 32 wherein said imaging mode is a 3D imaging mode.

35. The method according to claim 31 the steps further comprising:
positioning said matrix patch over at least one imaging target having an obstruction to visualize an image by repositioning sector scanning using said controls horizontally.

36. The method according to claim 31 the steps further comprising:
positioning said matrix patch over at least one imaging target having an obstruction to visualize an image by repositioning sector scanning using said controls vertically.

37. The method according to claim 31 the steps further comprising:
positioning said matrix patch over at least one imaging target having an obstruction to visualize an image by repositioning sector scanning using said controls to rotate.

38. The method according to claim 31 the steps further comprising:
positioning said matrix patch is positioned over at least one imaging target having an obstruction to visualize an image by repositioning sector scanning using said controls to tilt.

39. The method according to claim 31 the steps further comprising:
positioning said matrix patch over at least one imaging target having an obstruction to visualize an image by repositioning sector scanning using said controls to move the image along its x axis.

40. The method according to claim 31 the steps further comprising:
positioning said matrix patch over at least one imaging target having an obstruction to visualize an image by repositioning sector scanning using said controls to move the image along its y axis.

41. The method according to claim 32 the steps further comprising:
positioning said ultrasound imaging system in said imaging mode with said matrix patch over at least one imaging target to visualize an image by repositioning sector scanning using controls on said ultrasound imaging system to remove rib shadowing from said image.

42. The transducer and system according to claim 41 wherein said controls on said ultra imaging system includes a trackball for scrolling said image to a left or a right of said rib in order to position the image with the rib out of the way and soft key controls 54 on said ultra imaging system to provide various movement of the image such as tilt, elevation, biplane rotate, etc. for movement of said image from said rib.

43. The method according to claim 31 the steps further comprising:
positioning said ultrasound imaging system in an imaging mode with said matrix patch over at least one target to visualize at least one target by repositioning sector scanning using controls on said ultrasound imaging system.

44. The method according to claim 41 further comprising the steps of:
removing rib shadowing by operating said ultrasound imaging system in a 2D imaging mode with said matrix patch positioned over an imaging target and visualizing an image by repositioning sector scanning horizontally using controls on said ultrasound imaging system.

45. The method according to claim 44 wherein said controls on said ultra imaging system includes a trackball for scrolling said image to a left or a right of said rib in order to position the image with the rib out of the way and soft key controls on said ultra imaging system to provide various movement of the image such as tilt, elevation, biplane rotate, etc. for movement of said image from said rib.

46. The method according to claim 31 wherein said pad is a disposable pad.

47. The method according to claim 31 wherein said pad is a reusable pad.

48. The method according to claim 31 wherein said matrix array and said ultrasound imaging system are connected transducer wiring pad.

49. The method according to claim 31 wherein said matrix array and said ultrasound imaging system are connected by wireless technology.

50. The method according to claim 31 wherein said wireless technology is Bluetooth® technology.

51. The method according to claim 31 wherein said matrix array is formed as multiple pads for imaging.
